(19) United States
(12) Patent Application Publication
(10) Pub. No.: US 2023/0210814 A1
(43) Pub. Date: Jul. 6, 2023

(54) METHOD AND PHARMACEUTICAL COMPOSITION FOR INHIBITING CANCER METASTASIS

(71) Applicant: RAMOT AT TEL AVIV UNIVERSITY LTD., Tel Aviv (IL)

(72) Inventors: Neta EREZ, Tel Aviv (IL); Hila Doron, Tel Aviv (IL); Omer Adler, Tel Aviv (IL)

(21) Appl. No.: 18/110,960

(22) Filed: Feb. 17, 2023

Related U.S. Application Data
(63) Continuation of application No. PCT/IL21/51339, filed on Nov. 11, 2021.
(60) Provisional application No. 63/113,888, filed on Nov. 15, 2020.

(51) Int. Cl. A61K 31/366; C07K 16/32; A61P 35/04; A61P 25/00; G01N 33/50

(52) U.S. Cl. CPC A61K 31/366; C07K 16/32; A61P 35/04; A61P 25/00; G01N 33/5011; C07K 2317/76

(57) ABSTRACT

The invention provides a method for treating or preventing brain metastases comprising the step of administering to a patient in need a composition comprising a therapeutically effective amount of LCN2 Inhibitor, an agent that interferes in systemic LCN2 signaling pathways, or an agent that reduces LCN2 expression or any combination thereof.

### CROSS REFERENCE TO RELATED APPLICATIONS

This application is a Bypass Continuation of PCT Patent Application No. PCT/IL2021/051339 having International filing date of Nov. 11, 2021, which claims the benefit of priority of U.S. Provisional Patent Application No. 63/113,888, filed Nov. 15, 2020, the contents of which are all incorporated herein by reference in their entirety.

### BACKGROUND OF THE INVENTION

Cancer metastasis is one of the most important factors determining the prognosis of cancer patients and is the main process that determines death caused by cancer. In cancer therapies such as surgery, radiotherapy, chemotherapy and the like, a lot of efforts have been made to improve the survival of patients. The field of studying cancer metastasis is one of the last strategies to overcome cancer, and studies on cancer metastasis suppressors are essential for developing metastasis-suppressing drugs.

Brain metastases are more common than primary CNS tumors and confer grave prognosis on patients, with a median survival of less than one year.

Malignant melanoma is the deadliest skin cancer with rising incidence worldwide. Melanoma frequently metastasizes to the lungs, bone, liver and brain. Although the development of targeted therapies and immune checkpoint inhibitors has dramatically improved patient overall survival, brain metastases still pose an unmet clinical challenge. The microenvironment plays a crucial role in facilitating metastasis by promoting survival, colonization and proliferation of disseminated tumor cells to distant organs. Astrocytes are key components of the brain microenvironment. Neuroinflammation is a prominent feature of reactive astrocytes, characterized by the release of pro-inflammatory cytokines, increased blood-brain barrier permeability and immune cell infiltration, and is also a hallmark of brain metastatic niche formation. The mechanisms underlying survival and colonization of metastatic melanoma cells in the brain are poorly understood.

Accordingly, there is a great need in the art to identify therapeutic interventions to treat brain metastases.

### SUMMARY OF THE INVENTION

The experiments in the Examples section clearly show that LCN2 is a central player in facilitating brain metastasis, and a prognostic marker in human brain metastasis, linked with disease progression and poor survival. LCN2 mediates the intricate interactions between recruited innate immune cells and resident astrocytes in the brain metastatic niche that facilitate brain metastasis. The experiments further show that systemic LCN2 signaling derived from stromal cells in the primary tumor instigates pro-inflammatory activation of astrocytes. LCN2-activated astrocytes promoted the recruitment of immunosuppressive myeloid cells to the brain metastatic microenvironment, which then become a main source of LCN2 signaling. Functionally, genetic targeting of LCN2 resulted in attenuated neuroinflammation and decreased brain metastasis. Moreover, in human blood and tissue samples from patients with brain metastases from multiple cancer types, systemic LCN2 levels were strongly correlated with disease progression and poor survival, positioning LCN2 as a novel prognostic marker for brain metastasis.

In some embodiments, there is provided a method for treating or preventing brain metastases comprising the step of administering to a patient in need a composition comprising a therapeutically effective amount of LCN2 Inhibitor, an agent that interferes in systemic LCN2 signaling pathways, or an agent that reduces LCN2 expression or any combination thereof. In some embodiments, the systemic LCN2 signaling pathways instigate neuroinflammation.

In some embodiments, the LCN2 Inhibitor, the agent that interferes in systemic LCN2 signaling pathways, or the agent that reduces LCN2 expression, inhibits astrocytes activation.

In some embodiments, the agent that interferes in systemic LCN2 signaling pathways is an agent that suppresses downstream pathways of LCN2-mediated astrocyte activation. In some embodiments, the agent that suppresses downstream pathways of LCN2-mediated astrocyte activation suppresses JAK2-STAT3 and/or Rho-ROCK. The agent may be is some embodiments, a statin.

In some embodiments, the LCN2 inhibitor, the agent that interferes in systemic LCN2 signaling pathways and/or the agent that reduces LCN2 expression is a neutralizing antibody, a small molecule inhibitor or an antibody to the receptor, an aptamer, a small interfering RNA, a small internally segmented interfering RNA, a short hairpin RNA, a microRNA, and/or antisense oligonucleotide.

In some embodiments, the patient in need is a patient that suffers from melanoma, cutaneous malignant melanoma, melanoma tumorigenesis, breast cancer, lung cancer, melanoma, prostate cancer, colorectal cancer, bladder cancer, bone cancer, blood cancer, thyroid cancer, parathyroid cancer, bone marrow cancer, rectal cancer, throat cancer, laryngeal cancer, esophageal cancer, pancreatic cancer, gastric cancer, tongue cancer, skin cancer, brain tumor, uterine cancer, head or neck cancer, gallbladder cancer, oral cancer, colon cancer, anal cancer, central nervous system tumor, liver cancer, renal cell carcinoma and colorectal cancer.

In some embodiments, there is provided a method for identifying the suitability of a candidate compound or molecule that inhibits LCN2, interferes in systemic LCN2 signaling pathways, or reduces LCN2 expression, for treating brain metastasis comprising: contacting primary astrocytes with either RMS or sBT conditioned medium with or without the candidate compound or molecule; and comparing activity of astrocytes, wherein if the candidate compound or molecule reduces astrocytes activation in comparison to a control the candidate compound or molecule is suitable for treating the brain metastases.

In some embodiments, there is provided a method of determining the severity of brain metastases in a subject afflicted with melanoma, breast cancer or lung cancer comprising the step of comparing LCN2 levels in a blood of the subject with LCN2 levels of a normal healthy subject and/or to known LCN2 levels of patients with brain metastases, wherein overexpression of LCN2 determines that the patient has brain metastases and overexpression of LCN2 that is in the level of LCN2 of a patient with severe brain metastases indicates that the patient is at a severe stage of brain metastases.

In some embodiments, there is provided a method of determining the survival of a subject afflicted with melanoma, breast cancer or lung cancer with or without brain metastases comprising the step of comparing LCN2 levels in a blood of the subject with LCN2 levels of a normal healthy subject and/or severe patients, wherein overexpression of the LCN2 beyond the levels of a healthy normal subject determines that the patient has poor survival and overexpression of the LCN2 at the levels of severely ill melanoma, breast cancer or lung cancer patients with low survival determines that the survival of a subject is similar to the survival of severely ill melanoma, breast cancer or lung cancer patients.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A, 1B, 1C, 1D, 1E, 1F, 1G, 1H, 1I, 1J, 1K, 1L: Systemic LCN2 signaling is upregulated in plasma and CSF in brain metastases, and corelates with metastatic burden. FIG. 1A. Experimental scheme: mice were analyzed 18 days after intracardiac injection with BT-RMS or BT-EO771 cells. FIGS. 1B, 1F. LCN2 plasma levels measured by ELISA in mice injected as depicted in A. Dots represent individual mice, error bars represent SEM, (melanoma n=14, 20), (breast n=9, 20) (One-way ANOVA). FIGS. 1C, 1G. Pearson correlation analysis between LCN2 plasma levels and brain metastatic burden (% of CD45⁻ mCherry⁺/tdtomato⁺ tumor cells). FIGS. 1D, 1H. LCN2 CSF levels measured by ELISA in mice from A. Dots represent individual mice, error bars represent SEM, (melanoma n=10, 16), (breast n=7, 11) (One-way ANOVA). FIGS. 1E, 1I. Pearson correlation analysis for LCN2 in CSF and brain metastatic burden. FIGS. 1J-1L. ELISA assay for LCN2 plasma levels in human samples with BrM from patients with BrM: melanoma (n=3), breast (n=5), lung (n=6) or healthy controls (n=8). Dots represents individual patients, error bars represent SEM, (Student's t test).

FIGS. 2A, 2B, 2C, 2D, 2E, 2F, 2G, 2H, 2I, 2J, 2K, 2L, 2M: LCN2 signaling is functionally important for brain metastases formation and originates mainly from granulocytes and endothelial cells. FIG. 2A. Experimental scheme analyzed in B-F. FIG. 2B. Survival curve analysis of WT and LCN2^{-/-} mice injected intracardially with BT-RMS cells. Two independent experiment, n=20 per group (Kaplan-Meier curve, log-rank test). FIG. 2C. Brain macrometastases incidence defined by positive MRI and/or gross inspection (Analysis of contingency, Chi-square). FIGS. 2D, 2E. Representative MRI images and quantification of metastatic area, in WT and LCN2^{-/-} (n=9, n=10 respectively). Dots represent individual mice, error bars represent SEM (Student's t test). FIG. 2F. FACS analysis quantification of brain metastatic burden for mice in D. % CD45^{-/-} mCherry⁺ tumor cells/live cells. Dots represent individual mice, error bars represent SEM (one-way ANOVA). FIG. 2G. Experimental scheme analyzed in H-M: WT and LCN2^{-/-} BrM mice injected intracardially with BT-RMS or BT-EO771 cells 18 days after injection. FIGS. 2H, 2J. LCN2 ELISA in blood of mice from G. Dots represent individual mice, error bars represent SEM (melanoma n=20, 5), (breast n=20, 14). FIGS. 2I, 2K. LCN2 ELISA in CSF of mice from G. Dots represent individual mice, error bars represent SEM (melanoma: n=16, 9), (breast: n=11, 4). FIGS. 2L, 2M. qPCR analysis of LCN2 expression in FACS sorted cell population from brains of mice described in G, dots represent individual mice, error bars represents SEM (One-way ANOVA).

FIGS. 3A, 3B, 3C, 3D, 3E, 3F, 3G, 3H: Inflammatory activation of astrocytes is partially mediated by specific LCN2 receptor signaling. FIGS. 3A, 3B. qPCR analysis of SLC22A17 expression in FACS sorted cell populations from whole brains of mice with BrM following BT-RMS or BT-EO771 injection. Dots represents individual mice, error bars represent SEM (One-way ANOVA). FIG. 3C. Expression of SLC22A17 in bulk RNA-seq of different cell population isolated from samples of human BrM (Brain TIME dataset). Dots represents individual patients (One-way ANOVA). FIGS. 3D, 3E. Expression level of inflammatory gene signature measured by qPCR in RNA of FACS sorted astrocytes in vivo from WT or LCN2^{-/-} mice with BrM following BT-RMS or BT-EO771 injection. Heatmaps represent z-score of individual genes. FIGS. 3F-3H. Representative immunofluorescence staining in frozen sections of WT or LCN2^{-/-} mice with BrM. Co-localization of pP65 with astrocytes (GFAP) was quantified by GFAP MFI/pP65 MFI. Representative images are shown from n=3 mice per group. 10 fields×2 sections per mouse were analyzed (Student's t test).

FIGS. 4A, 4B, 4C, 4D: LCN2 signaling facilitates recruitment of immune suppressive myeloid cells into brain metastases. FIG. 4A. Immune profiling of CD11b⁺ myeloid cells by flow cytometry of BrM in WT or LCN2^{-/-} mice injected with BT-RMS cells. FIG. 4B. Immune profiling of CD11b⁺ myeloid cells by flow cytometry of BrM in WT or LCN2^{-/-} mice injected with BT-EO771 cells. FIGS. 4C, 4D. Heatmap showing z-score of immunosuppressive gene signature expression analyzed by qPCR of granulocytes isolated from WT or LCN2^{-/-} mice, following BT-RMS or BT-EO771 injection (Melanoma Ctrl n=5, WT n=6, LCN2^{-/-} n=6), (Breast Ctrl n=3, WT n=5, LCN2^{-/-} n=6).

FIGS. 5A, 5B, 5C, 5D, 5E, 5F, 5G: LCN2 signaling from bone marrow-derived cells plays a key functional role in facilitating brain metastasis. FIG. 5A. Experimental scheme analyzed in B-G, lethally eradiated WT recipient mice received WT or LCN2^{-/-} whole BM, injected with BT-RMS and analyzed. FIG. 5B. LCN2 ELISA in blood, one and two weeks following BMT. Dots represent individual mice, error bars represent SEM (One-way ANOVA). FIGS. 5C, 5D. Representative MRI images and quantification of metastatic area, 21 days post injection of BT-RMS cells (WT BM n=13, LCN2^{-/-} BM n=12), error bars represent SEM (Student's t test). FIG. 5E. Brain metastatic incidence quantification. Macrometastases were defined by positive MRI and flow cytometry detection. The cutoff for micrometastases was determined by % CD45⁻ mCherry⁺ tumor cells/live in normal WT mice. FIG. 5F. LCN2 ELISA in blood of mice at endpoint (One-way ANOVA). FIG. 5G. Immune profiling of CD11b⁺ myeloid cells by flow cytometry of mice injected with BT-RMS cells.

FIGS. 6A, 6B, 6C, 6D, 6E, 6F, 6G, 6H, 6I, 6J: Microenvironment-derived LCN2 signaling is operative in human patients and correlates with decreased survival in patients with brain metastases from melanoma, breast and lung primary origin. FIGS. 6A, 6B. Expression of LCN2 in bulk RNA-seq of different cell population isolated from samples of human BrM and gliomas (Brain TIME dataset), dots represent individual patients (One-way ANOVA). FIG. 6C, 6G. LCN2 ELISA in blood of patients with BrM from melanoma (n=9), lung (n=38) and healthy control samples (n=6) (Student's t test). FIG. 6D. Longitudinal follow-up of plasma LCN2 levels in patients with melanoma BrM following surgical resection. FIG. 6E. Pearson analysis for correlation between LCN2 plasma levels, measured at the patients last follow-up and overall survival (OS) in days for patients in (C,D). FIG. 6F. Survival curve analysis of patients with low vs. high LCN2 levels in patients from (C,D). The cutoff between high and low levels was defined as the median LCN2 level (Kaplan-Meier curve, log-rank test). FIG. 6H. Pearson analysis for the correlation between LCN2 plasma levels, measured prior to BrM resection, and the overall survival (OS) in days for patients in (G). FIG. 6I. 2-year survival curve analysis of patients with low vs. high LCN2 levels in patients from (G). The cutoff between high and low levels was defined as the median LCN2 level (Kaplan-Meier curve, log-rank test). FIG. 6J. 5-year survival curve analysis of patients with KPS score<70, stratified to low vs. high LCN2 levels in patients from (G).

FIGS. 7 (7A, 7B, 7C, 7D, 7E and 7F): Treatment with simvastatin or neutralizing antibody for LCN2 attenuates activation of astrocytes by melanoma CM. Adult primary astrocytes were incubated with RMS or sBT CM together with LCN2 neutralizing antibody or with simvastatin for 24 h. Astrocytes were then lysed and RNA extraction was performed followed by qPCR analysis for a gene signature of activated astrocytes, including LCN2 (FIG. 7A), Cp (FIG. 7B), Aspg (FIG. 7C), Steap4 (FIG. 7D), S1pr3 (FIG. 7E) and Osmr (FIG. 7F).

### DETAILED DESCRIPTION OF THE INVENTION

The Examples below show that LCN2 is a central factor in facilitating brain metastasis from multiple cancer types. Moreover, LCN2 is a novel diagnostic and prognostic factor in human patients, linked with disease progression and poor survival. Mechanistically, it was demonstrated that systemic LCN2 instigates neuroinflammation in the brain metastatic niche by activation of astrocytes, leading to recruitment of LCN2-producing granulocytes from the bone marrow to the brain metastatic microenvironment. These functions of LCN2 are critical for brain metastasis, as ablation of LCN2 in mice resulted in significant attenuation of brain metastases formation and improved survival.

The findings in mouse models of melanoma and breast cancer implicated systemic LCN2 as an inducer and potential prognostic marker of brain metastasis. Specifically, it was found that high levels of LCN2 in the CSF are a characteristic feature of brain metastasis, and are in correlation with brain metastatic burden.

Mechanistically, it was shown that LCN2 derived from stromal cells in the primary tumor give rise to high systemic levels of LCN2, conceivably instigating astrocyte activation. In the brain microenvironment, astrocytes respond to LCN2 signaling in a receptor-specific manner, resulting in activation of NF-κB and upregulation of pro-inflammatory signaling.

Within the recruited myeloid cells, granulocytes were the main source of LCN2 signaling, further augmenting astrocyte activation, neuroinflammation and metastatic growth. Adoptive BMT from LCN2^{-/-} mice to WT mice was sufficient to reduce metastatic burden in these mice, phenocopying LCN2^{-/-} mice, implicating granulocyte-derived LCN2 as a central player in orchestrating brain metastases formation. The findings shown herein elucidate for the first time a role for recruited granulocytes in astrocyte activation, and demonstrate the key functional importance of their LCN2-mediated signaling for brain metastatic growth.

The findings in mouse models, that systemic levels of LCN2 are in correlation with brain metastatic burden were supported by analysis of LCN2 levels in the blood of melanoma patients: high LCN2 blood levels upon initial diagnosis correlated with worse survival, and, strikingly, longitudinal follow-up of patients with brain metastasis revealed that elevation in their LCN2 blood levels co-insides with patient death. Taken together, the data in mouse models and in human patients suggest that systemic LCN2 has both a functional role in instigating a hospitable inflammatory niche, and a prognostic role, correlating with disease progression and outcome.

Patients with brain metastatic relapse have very poor prognosis, and their long-term (over two years) survival is usually negligible. As such, the clinical decision whether to operate on brain metastases is complicated, and dependent on prognosis. Our data, combining LCN2 levels with KPS score provide a strong tool to predict long term survival which is not revealed by each factor separately. Thus, the results demonstrated herein implicating LCN2 as a predictive factor in patient stratification provide a novel tool to assess prognosis and instruct clinical decision making.

In summary, the experiments of the invention position LCN2 as a key factor in the crossroad of the intricate interactions between systemic inflammatory mediators and the metastatic microenvironment, and provides insights into the reciprocal communication between glial cells and recruited innate immune cells in the brain metastatic niche. The functional and prognostic aspects of LCN2 that were identified in brain metastasis suggest that targeting LCN2 is an effective therapeutic target for inhibition or prevention of brain metastatic relapse.

In some embodiments of the invention, there is provided a method for treating or preventing brain metastases comprising the step of administrating to a patient in need a composition comprising a therapeutically effective amount of LCN2 Inhibitor, an agent that interferes in systemic LCN2 signaling pathways, and/or an agent that reduces LCN2 expression.

In some embodiments, the systemic LCN2 signaling pathways instigate neuroinflammation. In some embodiments, Lipocalin-2 (LCN2) is a 25 kDa secreted glycoprotein known for sequestering iron as a physiological response of fighting bacterial infections. LCN2 was also shown to be a pro-inflammatory factor, overexpressed in various malignancies. More importantly, LCN2 is a known activator of astrocytes, implicated in numerous CNS pathologies. However, the role of LCN2 in melanoma is largely unexplored.

In some embodiments, the LCN2 inhibitor, the agent that interferes in systemic LCN2 signaling pathways and/or the agent that reduces LCN2 expression is a neutralizing antibody, small molecule inhibitor, or an antibody to the receptor.

In some embodiments, the LCN2 inhibitor, the agent that interferes in systemic LCN2 signaling pathways and/or the agent that reduces LCN2 expression, is a specific antibody, aptamer, small interfering RNA, small internally segmented interfering RNA, short hairpin RNA, microRNA, and/or antisense oligonucleotide.

In some embodiments, the agent that interferes in systemic LCN2 signaling pathways is an agent that suppresses downstream pathways of LCN2-mediated astrocyte activation. In some embodiments, the agent that suppresses downstream pathways of LCN2-mediated astrocyte activation suppresses JAK2-STAT3 and Rho-ROCK is a statin.

In some embodiments, the statin is simvastatin. In some embodiments the statin is one or more of simvastatin, atorvastatin, fluvastatin, lovastatin, pitavastatin, pravastatin and rosuvastatin.

In some embodiments, the patient in need is a patient having cancer that can result in metastasis, or cancer resulting from metastasis. In some embodiments the patient in need is a patient afflicted with a brain metastasis or at a risk of being afflicted with brain metastasis. In some embodiment, the patient in need is a patient afflicted with a brain metastasis that are not yet detectable by using the current methods of detection. In some embodiments, the patient may have a micrometastatic tumor, wherein the tumor is too small to be visualized by radiological means. It can be a visible metastatic tumor, wherein the tumor is large enough to be discernable by clinical radiological means, such as magnetic resonance imaging, computerized tomography, or positron emission tomography. The metastatic lesions are distinct from metastatic cancer cells in the systemic circulation and single cancer cells extravasating into brain tissue or quiescently residing therein. The brain metastases can be progressive or stable, as assessed by a method, such as MM, CT, proliferation marker expression, and the like. The term “micrometastasis” as used herein is preferably defined as a group of confluent cancer cells measuring from greater than 0.2 mm and/or having greater than 200 cells to 2 mm in maximum width. Micrometastasis is generally not visible in standard contrast MRI imaging or other clinical imaging techniques. However, in certain cancers, radioactive antibodies directed to tumor selective antigens (e.g., Her2 for breast cancer metastasis) allows for visualization of micrometastasis. Other indirect detection methods include contrast media leakage at brain micrometastasis sites due to VEGF induced vascular leakage. More sensitive imaging techniques may also be applied to detect micrometastases.

In some embodiments, the patient in need is a patient suffering from metastatic malignant melanoma.

In some embodiments, the cancer that can result in metastases is any one selected from the group consisting of breast cancer, lung cancer, melanoma, prostate cancer, colorectal cancer, bladder cancer, bone cancer, blood cancer, thyroid cancer, parathyroid cancer, bone marrow cancer, rectal cancer, throat cancer, laryngeal cancer, esophageal cancer, pancreatic cancer, gastric cancer, tongue cancer, skin cancer, brain tumor, uterine cancer, head or neck cancer, gallbladder cancer, oral cancer, colon cancer, anal cancer, central nervous system tumor, liver cancer, renal cell carcinoma and colorectal cancer.

In some embodiments, the method and/or the composition of the invention confer healthy longevity and/or tumor resistance or metastasis resistance to the subject.

In some embodiments, the LCN2 inhibitor, the agent that interferes in systemic LCN2 signaling pathways, and/or the agent that reduces LCN2 expression, reduces the number of proliferating cells in the brain metastasis and/or increases the number of apoptotic cells in the brain metastasis. In some embodiments, the LCN2 inhibitor, the agent that interferes in systemic LCN2 signaling pathways and/or the agent that reduces LCN2 expression reduces, slows, delays or prevents the metastasis of the cancer.

As shown in the examples, that follow, the inventors have discovered that plasma levels of LCN2 increase in melanoma-bearing mice and in human patients with melanoma brain metastasis. Moreover, LCN2 is overexpressed in cells in the microenvironment of primary melanoma in mice.

These findings suggest that melanoma and stroma-derived LCN2 facilitates brain tropism and metastases formation by activating astrocytes and instigating neuroinflammation. In addition, it was shown that LCN2 high levels in serum are correlated with worse prognosis, and it is involved in tumor-promoting neuroinflammation in brain, thus, in some embodiments, LCN2 may be a diagnostic marker, a prognostic marker, a marker for studying the efficiency of a drug and further it may be target for therapeutic intervention in human metastases and in particular in brain metastasis.

In some embodiments of the invention, there is provided a method for identifying the suitability of a candidate compound or molecule that inhibits LCN2, interferes in systemic LCN2 signaling pathways, and/or reduces LCN2 expression, for treating brain metastasis comprising: contacting primary astrocytes with either RMS or sBT conditioned medium with or without the candidate compound or molecule; and comparing activity of astrocytes, wherein if the candidate compound or molecule reduces astrocytes activation in comparison to the control (i.e. without the candidate compound or molecules) may be suitable for treating the brain metastases.

In some embodiments, LCN2 inhibitors may be identified by their ability to suppress downstream pathways of LCN2-mediated astrocyte activation including JAK2-STAT3 and Rho-ROCK. This may be done by simple tests such as Western blot analysis for STAT3 phosphorylation.

In some embodiments, since it was shown herein that LCN2−/− mice are deficient in their ability to recruit immune cells (specifically—granulocytes) to brain metastasis, immune cell transwell migration assays in vitro with/without the agent which is assessed for its LCN2 inhibiting property will be performed.

In some embodiments, the step of contacting astrocytes with either RMS or sBT conditioned medium (CM) with or without the candidate compound or molecule is for between an hour to 48 hours.

In some embodiments, the activation of the astrocytes is assessed by analyzing the expression of a pan reactive astrocyte gene signature, wherein if the activation is attenuated by the candidate compound or molecule in comparison to control (astrocytes that were incubated with the RMS or sBT conditioned medium (CM) only), the candidate compound or molecule that inhibits LCN2, interferes in systemic LCN2 signaling pathways, or reduces LCN2 expression, is suitable for treating brain metastases.

In some embodiments, there is provided a method of determining the severity of brain metastases in a subject afflicted with melanoma, breast cancer or lung cancer comprising the step of comparing LCN2 levels in a blood of the subject with LCN2 levels of a normal healthy subject and/or to known LCN2 levels of patients with brain metastases, wherein overexpression of LCN2 determines that the patient has brain metastases and overexpression of LCN2 that is in the level of LCN2 of a patient with severe brain metastases indicates that the patient is at a severe stage of brain metastases.

In some embodiments, there is provided a method of determining the survival of a subject afflicted with melanoma, breast cancer or lung cancer with or without brain metastases comprising the step of comparing LCN2 levels in a blood of the subject with LCN2 levels of a normal healthy subject and/or severe patients, wherein overexpression of the LCN2 beyond the levels of a healthy normal subject determines that the patient has poor survival and overexpression at the levels of severely ill melanoma patients with low survival determines that the survival of a subject is similar to the survival of severely ill melanoma, breast cancer or lung cancer patients.

In some embodiments, the “normal” level or the “level/s of a normal healthy subject” of expression of an LCN2 biomarker is the level of expression of the biomarker in cells of a subject, e.g., a human patient, not afflicted with a cancer. An “over-expression” or “significantly higher level of expression” of the LCN2 biomarker refers to an expression level in a test sample that is greater than the standard error of the assay employed to assess expression, and is at least 10%, or 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the LCN2 biomarker in a control sample (e.g., sample from a healthy subject not having the biomarker associated disease) or the average expression level of the biomarker in several control samples.

An “over-expression” or “significantly higher level of expression” of a biomarker refers to an expression level in a test sample that is greater than the standard error of the assay employed to assess expression, and is at least 10%, or 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) or the average expression level of the biomarker in several control samples. In some embodiments, there is provided a database containing data of LCN2 levels from subjects with brain metastases at different stages and their survival, which enables estimation of the survival of the assessed patient.

Another aspect of the invention pertains to monitoring the efficiency of agents (e.g., drugs, compounds, and small molecules) on the expression or activity of LCN2. The diagnostic methods described herein can furthermore be utilized to identify subjects having or that are at risk of developing brain metastases that are likely or unlikely to be responsive LCN2 inhibitor therapy, to agent that interferes in systemic LCN2 signaling pathways, or to agent that reduces the expression of the LCN2. Furthermore, the prognostic assays described herein can be used to determine whether a subject can be administered with an LCN2 inhibitor therapy, an agent that interferes in systemic LCN2 signaling pathways and/or an agent that reduces the expression of the LCN2 (e.g., an agonist, antagonist, peptidomimetic, polypeptide, peptide, nucleic acid, small molecule, or other drug candidate) to treat brain metastases associated with the LCN2 expression or activity.

In some embodiments of the invention, there is provided a kit for measuring LCN2 levels in a blood of a subject comprising means for measuring LCN2 levels and a leaflet with normal level of a healthy control and/or leaflet containing data of LCN2 levels from subjects with brain metastases at different stages and their survival, which enables estimation of the survival of the assessed patient.

In some embodiments of the invention there is provided a composition comprising a therapeutically effective amount of one or more of an inhibitor of LCN2 and/or an agent that interferes in systemic LCN2 signaling pathways and/or an agent which reduces LCN2 expression. In some embodiments, the composition is a pharmaceutical composition, such as compositions that are suitable for administration to animals (e.g., mammals, primates, monkeys, humans, canine, feline, porcine, mice, rabbits, or rats). In some embodiments, the pharmaceutical composition is non-toxic, does not cause side effects, or both. In some embodiments, there may be inherent side effects (e.g., it may harm the patient or may be toxic or harmful to some degree in some patients).

The term “therapeutically effective amount” means an amount effective to achieve a desired and/or beneficial effect. An effective amount can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement for the presence or the level of the brain metastases or by measuring LCN2 levels in the plasma or CSF.

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, ameliorating symptoms, or otherwise reducing the risk of developing a particular condition. As used herein, reference to “treating” an animal includes but is not limited to prophylactic treatment for developing metastases or increasing the growth of metastases and therapeutic treatment for reducing their amount, spread or growth or limiting the rate of increase of tumor metastases. Any of the compositions (e.g., pharmaceutical compositions) described herein can be used to treat an animal.

In some embodiments, the treatment of the invention can be combined with one or more other treatments. For example, for treatment of metastatic melanoma, use of surgery, isolated limb perfusion, regional chemotherapy infusion (with e.g., decarbazine or cisplatin), radiation therapy, immunotherapy (e.g., treatment with antibodies against GD2 and GD3 gangliosides), intralesional immunotherapy, systemic chemotherapy, hyperthermia, systemic immunotherapy, tumor vaccines, or combinations thereof can be further combined with the LCN2 inhibitor and/or the agent that reduces expression of LCN2.

In some embodiments, the one or more LCN2 inhibitor or agent that reduces LCN2 expression is in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more pharmaceutical excipient or carrier. A “pharmaceutical excipient or carrier” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof.

In some embodiments, the composition or pharmaceutical composition comprises at least one active ingredient which can be administered to an animal (e.g., mammals, primates, monkeys, or humans) in an amount of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some animals (e.g., mammals, mice, rabbits, feline, porcine, or canine) can be administered a dosage of about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, it is possible to employ many concentrations in the methods of the present invention, and using, in part, the guidance provided herein, one could adjust and test any number of concentrations in order to find one that achieves the desired result in a given circumstance.

In some embodiments, the compositions can include a unit dose of the LCN2 inhibitor, the agent that interferes in systemic LCN2 signaling pathways, and/or the agent that reduces the expression of LCN2 in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

The route of administration of the LCN2 inhibitor, the agent that interferes in systemic LCN2 signaling pathways and/or agent that reduces the expression of LCN2, can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease (e.g., cancer), and the severity of the disease (e.g., stage or severity of cancer). Further, combinations of administration routes can be administered, as desired.

### EXAMPLES

The experiments and the results shown here clearly demonstrate that LCN2 is a central player in facilitating brain metastasis, and a prognostic marker in human brain metastasis, linked with disease progression and poor survival. LCN2 mediates the intricate interactions between recruited innate immune cells and resident astrocytes in the brain metastatic niche that facilitate brain metastasis. that systemic LCN2 signaling derived from stromal cells in the primary tumor instigates pro-inflammatory activation of astrocytes. LCN2-activated astrocytes promoted the recruitment of immunosuppressive myeloid cells to the brain metastatic microenvironment, which then become a main source of LCN2 signaling. Functionally, genetic targeting of LCN2 resulted in attenuated neuroinflammation and decreased brain metastasis. Moreover, in human blood and tissue samples from patients with brain metastases from multiple cancer types, systemic LCN2 levels were strongly correlated with disease progression and poor survival, positioning LCN2 as a novel prognostic marker for brain metastasis.

Methods

Mice

All animals were maintained within the Tel Aviv University Specific Pathogen Free (SPF) Facility. All Animal procedures included in the study were granted ethical approval by the Tel Aviv University Institutional Animal Care and Use Committee. B6.129P2-Lcn2tm1Aade/AkiJ (LCN2^{-/-}) were purchased from The Jackson Laboratory. Non-transgenic C57BL/6 mice were purchased from Harlan, Israel. Mice were used for experiments at 6-10 weeks of age, unless otherwise stated.

Human Samples

Human patient blood and tissue samples were collected with written informed consent.

Cell Lines

RMS (Ret-melanoma sorted) cells (19) and their derivative BT-RMS (13) were grown in RPMI media. E0771 derivative (BT-EO771) were generated by two cycles of in vivo selection using intra-cardiac injection, isolation, culture and re-injection of brain metastatic cells. Cells were grown in RPMI media. C166 endothelial cells were purchased from ATCC (ATCC® CRL-2581™), and grown in supplemented RPMI media. Cell lines were not authenticated in our laboratory. All cell lines were grown at 37° C. and 5% CO₂, and routinely tested for Mycoplasma.

Primary Cells

Dermal Fibroblasts (DFs) were isolated from ears of 8-12 weeks old male CS7BL/6J as previously described (46). All experiments were performed with low passage (p2-4) fibroblasts.

Adult Astrocytes were isolated from 6-8 weeks old CS7BL/6 and LCN2^{-/-} mice as previously described (13). Cells were cultured in 10% FCS RPMI media supplemented with astrocyte growth supplement (1852-scl, ScienCell). All experiments were performed with low passage (p2-4) primary cells.

Bone marrow-derived myeloid cells (BMDM) were isolated from the femur and tibia of 8-weeks old mice. Cells were cultured 6-8 days with media supplemented with 20 ng/ml recombinant mouse M-CSF (Peprotech, IL) and 0.1 mM non-essential amino acids.

Tissue Dissociation

Brains: mice were intracardiaclly perfused with cold PBS, their brains were harvested, minced, and dissociated using Papaine (LS004182, Worthington Biochemical Corporation), TrypLE (12604013, Thermo Fisher) and DNase (LS002007, Worthington Biochemical Corporation). RBC were lysed using NaCl hypotonic solution. Demyelination was achieved using percoll (P4937-500 ML, Sigma-Aldrich).

Primary tumors: resected melanoma and breast tumors were minced, and dissociated using Collagenase type 2 (LS004177, Worthington Biochemical Corporation) and Dispase type 2 (4942078001, Sigma-Aldrich). RBC were lysed using NaCl hypotonic solution.

ELISA

Mice: ELISA for mLCN2 was performed using R&D Systems; DY1857 commercial kit, according to manufacturer's protocol. Plasma samples were diluted 1:2000, CSF samples were diluted 1:40, CM samples were not diluted.

Human: ELISA for hLCN2 was performed using R&D Systems; DY1757 commercial kit, according to manufacturer's protocol. Blood samples were diluted 1:200.

Cerebrospinal Fluid Collection

Approximately 5 μL of cerebrospinal fluid (CSF) samples were obtained from the cisterna magna of mice brains. Samples were stored at −80° C. Only blood-free samples were analyzed.

Flow Cytometry

Single-cell suspensions were incubated with: anti-CD45-BV650 (BioLegened, BLG-103151), anti-CD11b-PeCy7 (BioLegend, BLG-101215), anti-Ly6G-APC (BioLegend, 127614), anti-Ly6C-FITC (BioLegend, 128006), and DAPI (MBD0015; Sigma-Aldrich). Samples were analyzed with Cytoflex LX, BECKMAN COULTER.

FACS Sorting

Single-cell suspensions of mouse brains were stained with the following anti-mouse antibodies: anti-CD45-BV650 (BioLegend, BLG-103151), anti-CD11b-PerCP-Cy5.5 (eBioscience, 45-0112), anti-Ly6G-APC-Cy7 (BioLegend, 127624), anti-Ly6C-FITC (BioLegend, 128006), anti-ACSA2-APC (130-102-315, Miltenyi Biotec), anti-CD31-PE-Cy7 (eBioscience, 25-0311), and DAPI (MBD0015; Sigma-Aldrich). Cancer cells were labeled with mCherry (melanoma) or tdTomato (breast). Different cell populations were isolated according to the gating strategy presented in the supplementary figures. Sorting was performed with BD FACSAria™ III Cell Sorter, BD Biosciences.

RNA Isolation and qRT-PCR

RNA from sorted cells was isolated using the EZ-RNAII Kit (20-410-100, biological industries). RNA from in vitro experiments and from total primary tumors was isolated using the PureLink RNA Mini Kit (Invitrogen; 12183018A). cDNA synthesis was conducted using qScript cDNA Syntesis Kit (Quanta, 95047-100). qRT-PCR were conducted using PerfeCTa SYBR Green Fastmix ROX (Quanta, 95073-012). Expression results were normalized to Gusb, Gapdh, or Ubc and to controls. RQ (2^{-ΔΔCt}) was calculated.

Immunostaining

Processing of mouse tissue: Brains were harvested, washed in PBS, examined by gross inspection for metastatic lesions and incubated for 5 h in 4% PFA (Electron Microscopy Sciences) and transferred to 1% PFA overnight. Brains were incubated in 0.5M sucrose for 1 h, then in 1M sucrose overnight. All incubations were performed at 4° C. Brains were embedded in Optimal Cutting Temperature compound (OCT, Tissue-Tek) on dry ice, then stored at −80° C.

Processing of human tissue: Resected brain metastases were immediately frozen and maintained in liquid nitrogen at the Rabin Medical Center, Beilinson Hospital BioBank. Tissues were collected and processed as described above for mice tissue.

10 μm serial sections were cut using a cryostat (CM1950, Leica), and slides were stored at −80° C.

Frozen brain tissue sections were incubated at 60° C. for 30 min, washed with PBS-T, then blocked with PBS with 1% BSA and 5% donkey serum for 30 min. Slides were incubated over night at 4° C. with the following primary antibodies: Mouse—rabbit anti-mouse GFAP 1:800 (Z-0334, Dako), rabbit anti-mouse IBA-1 1:200 (NBP2-19019, Novus), rabbit anti-mouse VWF 1:500 (ab6994, Abcam), rat anti-mouse Ly6G 1:500 (127601, Biolegend), goat anti-mouse LCN2 1:100 (AF1857, R&D), rabbit anti-mouse pP65 1:800 (#3033, Cell Signaling). Human—chicken anti-human GFAP 1:1000 (ab9377, Abcam), goat anti-human LCN2 1:100 (AF-1757, R&D), rabbit anti-human pan-cytokeratin 1:500 (ab9377, Abcam), mouse anti-human Melanoma 1:200 (ab732, Abcam), mouse anti-human CD66B 1:500 (G10F5, Novus). Slides were washed with PBS-T, and incubated for 1 h at RT with the following fluorescently-conjugated secondary antibodies: donkey anti-rabbit AF647 (711-605-152, Jackson), donkey anti-goat Dylight-488 (705-486-147, Jackson), donkey anti-rat AF647 (712-605-153, Jackson), donkey anti-mouse AF647 (712-605-153, Jackson) diluted 1:200. Stained slides were mounted with DAPI Fluoromount-G (0100-20, Southern Biotech), left to dry for 2 h at RT and stored at 4° C. Images were acquired using the confocal ZEISS LSM800 platform, with a ×40/1.4 oil objective or a ×20/0.75 air objective, or by using the Leica Aperio VERSA slide scanner with a ×20 magnification. All images were analyzed using ImageJ software.

Orthotopic Tumor Transplantations

Melanoma: 5×10⁵ low passage BT-RMS cells were inoculated intradermally as previously described (19). Tumor volumes were calculated using the formula X²×Y×0.5 (X-smaller diameter, Y-larger diameter).

Breast: 2×10⁵ BT-EO771 cells were inoculated into the mammary glands as previously described (47). Tumors were resected 3 weeks following injection.

Intracardiac Injections

8-week-old C57BL/6 or LCN2^{-/-} mice were anesthetized with Ketamine/Xylazine and injected with 1×10⁵ BT-RMS, and females with 2×10⁵ BT-EO771 cells in 50 μl PBS into the left ventricle of the heart under an ultrasound guidance. Mice were weighed every other day and monitored for neurological symptoms.

Bone Marrow Transplantations (BMT)

8-week-old male C57BL/6 WT mice were lethally irradiated using an x-ray machine (160HF; Philips) at a total dose of 9 Gy. 24 h post-irradiation, mice were injected intra-venously (IV) with 2.0×10⁶ unfractionated BM cells harvested aseptically from flushed femur and tibia of age-matched C57BL/6 WT or LCN2^{-/-} male mice. Following transplantation, mice received antibiotics for 4 wk in drinking water (Enrofloxacin; 0.2 mg/ml). To ensure radiation lethality, one mouse of each group was irradiated without transplantation. Three weeks post transplantation, mice were anesthetized with Ketamine/Xylazine and injected intra-cardially with 1×10⁵ BT-RMS cells. Mice were weighed every other day. 17 days following injections, mice underwent MRI imaging and euthanized 4 days later.

MRI Imaging

Mice were anesthetized by isoflurane. T1 weighted images with contrast agent (Magnetol, Gd-DTPA, Soreq M.R.C. Israel Radiopharmaceuticals) were taken by 4.7 T MRI-MRS 4000™ (MR solutions). Tumor volume was calculated using Radiant Dicom Viewer 2020.1.1.

Human RNA-Seq Data Analysis

The complete raw count matrix of all sorted populations and the full clinical annotation was downloaded as csv files from the publicly available Brain TIME dataset (10). Expression level of specific genes of interest was analyzed across all available different cell populations.

Statistical Analyses

Statistical analyses were performed using GraphPad Prism software. All tests were two-tailed. For in vitro experiments data represent mean and SD of at least three separate biological repeats. For in vivo experiments data represent mean and SEM of at least two separate biological repeats. For data with normal distribution, Student's t test/ANOVA (One-way/Two-way/Repeated measures) was used according to the experimental setup. For data with non-normal distribution, Kruskal-Wallis test was used. Correlation analysis were performed with Fisher exact test (2×2 contingency table). P value of ≤0.05 was considered statistically significant.

### Example 1

Systemic LCN2 is Associated with Melanoma and Breast Cancer Brain Metastasis

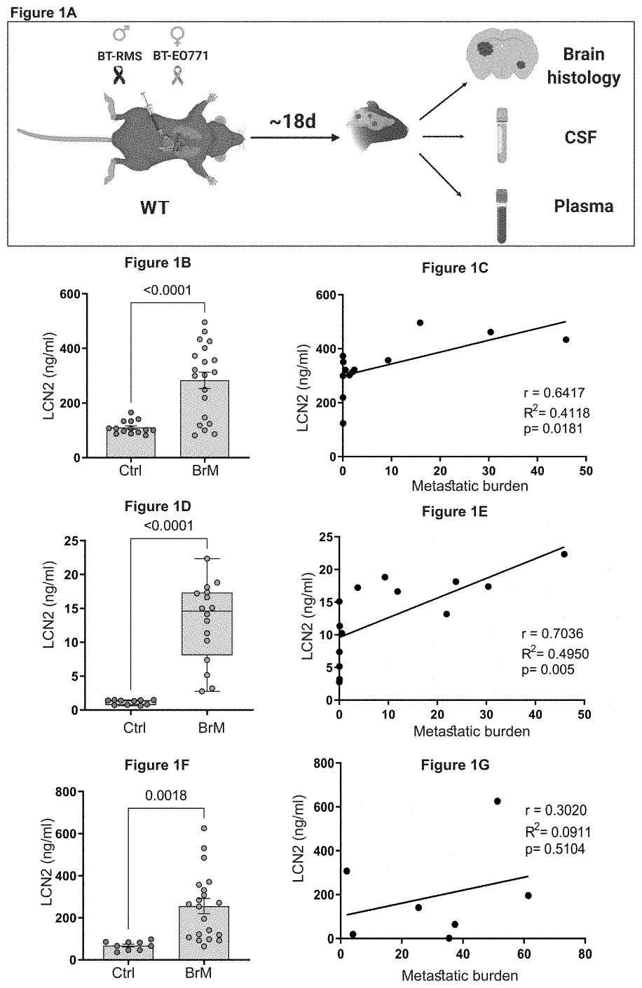

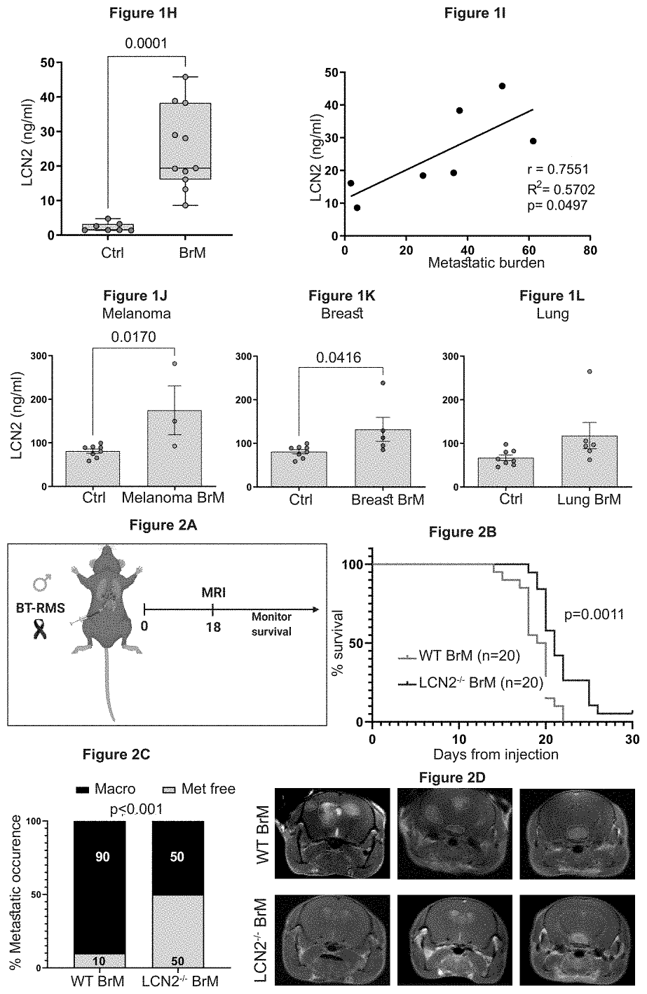

To assess systemic levels of LCN2, models of experimental brain metastasis of melanoma and breast cancer were utilized (FIG. 1A). LCN2 protein levels in plasma and in cerebrospinal fluid (CSF) of mice with brain metastasis were analyzed, and it found that LCN2 was systemically upregulated in blood and CSF of metastases-bearing mice in both melanoma (FIG. 1B, 1D) and breast cancer-derived metastases (FIG. 1F,1H), compared with healthy mice. Moreover, the levels of LCN2 in both blood and CSF correlated with brain metastatic burden (FIG. 1C,1E,1G,1I), suggesting a link with disease progression. To validate these findings in human patients, LCN2 levels in blood samples from human patients with brain metastasis from melanoma, breast cancer or lung carcinoma were assessed. The results confirmed that LCN2 is systemically upregulated in patients with brain metastasis compared with controls (FIG. 1J-1L). Taken together, these findings suggest a functional role for LCN2 in facilitating brain metastasis.

### Example 2

LCN2 is Functionally Important for Brain Metastasis

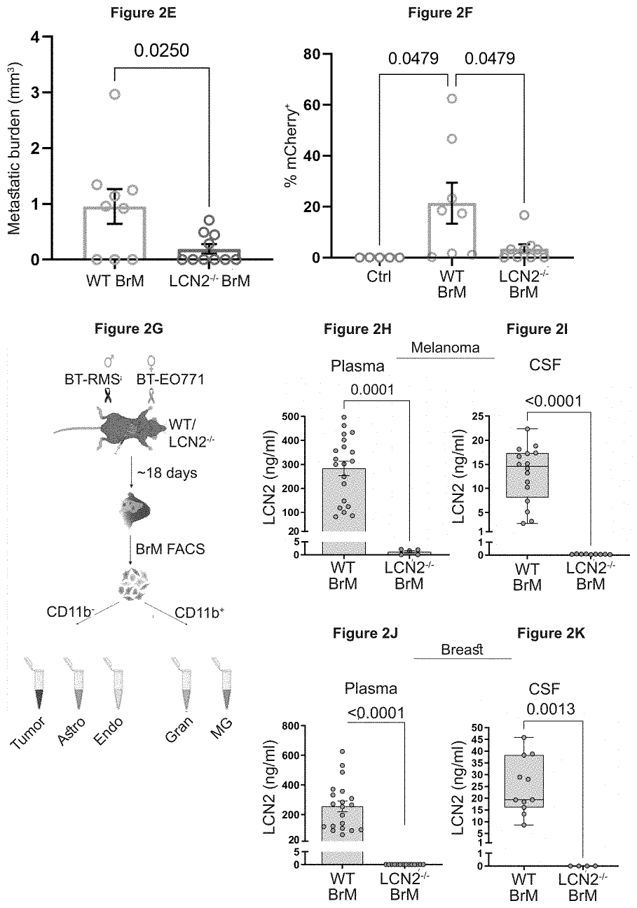

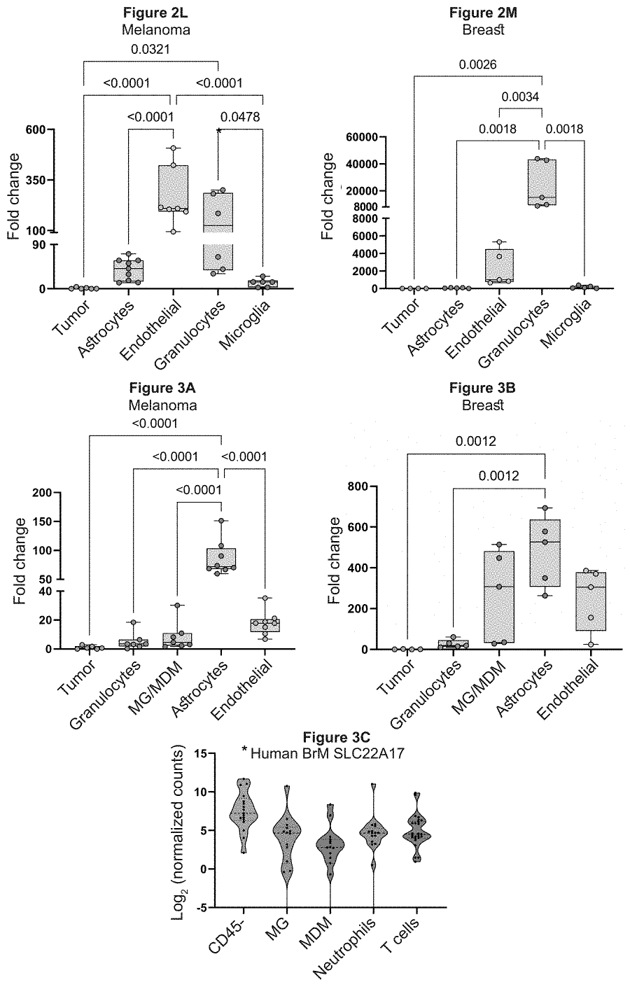

Based on the high levels of LCN2 in mice with brain metastases, it was hypothesized that LCN2 may be functionally important for brain metastatic growth. To test this, brain metastases from melanoma and breast cancer in WT or LCN2^{-/-} mice (FIG. 2A,2G) were analyzed. Strikingly, it was found that the survival of WT mice was reduced compared with LCN2^{-/-} mice, consistent with a dramatic decrease in the percentage of mice with macro-metastases in LCN2^{-/-} mice (FIG. 2B,2C). Moreover, intravital analysis of metastatic burden by MRI imaging confirmed that WT mice had more metastatic lesions (FIG. 2D,2E). This was further supported by quantification of mCherry⁺ tumor cells in brains of WT or LCN2^{-/-} mice (FIG. 2F), implicating LCN2 as an important mediator of brain metastases formation. Analysis of brain metastases in WT or LCN2^{-/-} mice injected with breast cancer cells indicated no significant differences in survival and brain metastatic burden (not shown), suggesting that additional pathways are operative in breast cancer metastasis.

To get mechanistic insight on the role of LCN2 signaling in brain metastasis, blood, CSF and multiple cell populations were isolated from the brain microenvironment in metastases-bearing WT or LCN2^{-/-} mice: mCherry/tdTomato⁺ cancer cells, CD45⁻ACSA2⁺ astrocytes, CD45⁻CD31⁺ endothelial cells, Ly6G⁺Ly6C^{int} granulocytes and Ly6G⁻Ly6C⁻ microglia/monocyte-derived macrophages (MG/MDM) (FIG. 2G). In agreement with the findings from primary tumor analysis, the results confirmed that the source of LCN2 is almost exclusively host-derived, in both melanoma and breast cancer brain metastasis (FIG. 2H-2K). Moreover, detailed analysis of LCN2 expression in specific cell types from melanoma or breast cancer brain metastases indicated that granulocytes and endothelial cells are the main source of LCN2 (FIG. 2L,2M). Thus, LCN2 in brain metastases is secreted by recruited granulocytes and brain endothelial cells, and is functionally important for brain metastases formation.

### Example 3

LCN2 Mediates Inflammatory Activation of Astrocytes and Myeloid Cell Recruitment in the Brain Metastatic Microenvironment

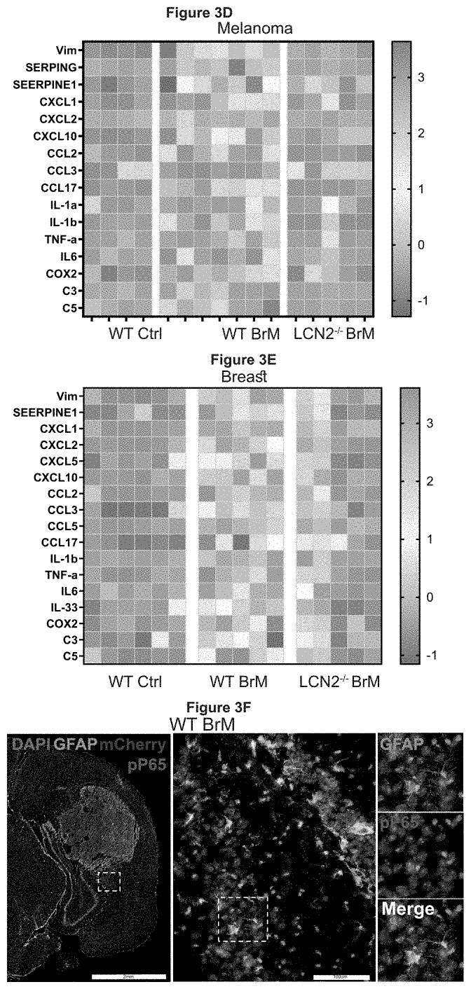

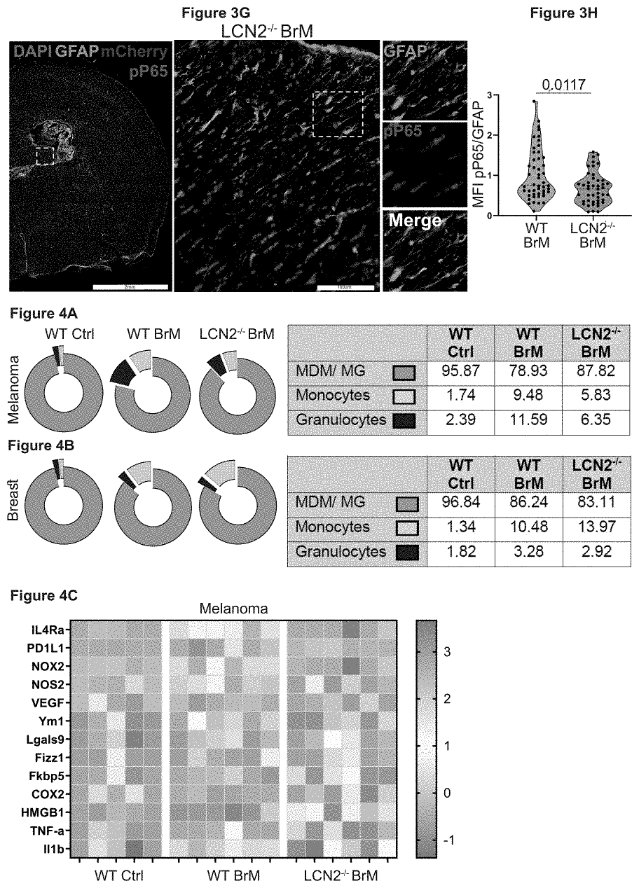

To decipher the mechanism by which LCN2 facilitates brain metastatic growth cells were from the brain metastatic microenvironment as above, and the expression of SLC22A17, the specific LCN2 receptor was analyzed. In both melanoma and breast cancer metastases, the highest expression of the LCN2 receptor was in astrocytes, and to a lesser extent in endothelial cells and microglia (FIG. 3A, 3B). Moreover, the expression of the LCN2 receptor in a dataset of human patients with brain metastases from melanoma, lung and breast cancer, as well as in primary brain tumors was analyzed, and found that its expression was highest in CD45⁻ stromal cells (FIG. 3C). Since LCN2 is a known activator of astrocytes and a mediator of neuroinflammation, the activation and inflammatory status of metastases-associated astrocytes isolated from brain metastases of WT or LCN2^{-/-} mice was assessed. Analysis of the results indicated that LCN2 plays an important role in inflammatory activation of metastases-associated astrocytes: the expression of multiple cytokines and chemokines was significantly reduced in astrocytes isolated from LCN2^{-/-} mice, in both melanoma and breast cancer metastasis (FIG. 3D, 3E). Of note, these changes in astrocytes were metastasis-specific, as lack of LCN2 did not affect the activation status of normal astrocyte (not shown). Furthermore, LCN2-mediated signaling in brain were receptor-specific, as microglia cells, which express low levels of the LCN2 receptor did not exhibit differential activation in WT vs. LCN2^{-/-} mice.

Many of the cytokines and chemokines that were upregulated in metastases-associated astrocytes are known target genes of the NF-κB transcription factor (CXCL1, CXCL2, IL-1β COX-2, IL-6). The effect of LCN2 on NF-κB activation in astrocytes was assessed. Immunostaining for the phosphorylated form of the NF-κB subunit p65 (RelA) was performed. When active, the NF-κB heterodimer (RelA-p50) translocates to the nucleus, where it activates the transcription of its target genes (28,29). pP65 was highly expressed in astrocytes in WT brain metastases, but not in LCN2^{-/-} brain metastases (FIG. 3F-3H). Taken together, these results demonstrate that LCN2, secreted by recruited myeloid cells in the brain metastatic microenvironment instigates pro-inflammatory signaling in astrocytes via receptor-mediated NF-κB activation.

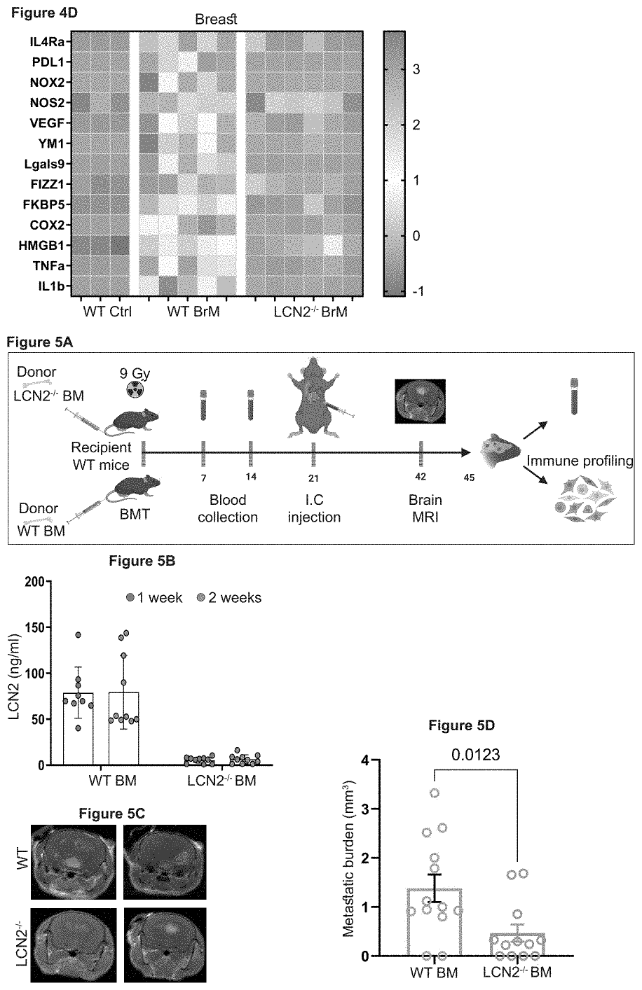

Since many of the pro-inflammatory genes activated in metastases-associated astrocytes are known chemoattractants for innate immune cells, it was hypothesized that the mechanism by which LCN2 signaling facilitates brain metastasis includes the recruitment of tumor-promoting immune cells. To test this, the immune milieu in brains of WT or LCN2^{-/-} mice with brain metastases was analyzed. Analysis of myeloid cells in melanoma brain metastases revealed that while both Ly6G⁺Ly6C^{int} granulocytes and Ly6C⁺Ly6G⁻ monocytes were elevated, their recruitment was inhibited in brain metastases of LCN2^{-/-} mice (FIG. 4A), implying that LCN2 is functionally important for their recruitment. Of note, analysis of normal brains from WT or LCN2^{-/-} mice did not show significant differences in the presence of myeloid cells (not shown), indicating that LCN2-driven recruitment of myeloid cells is specifically functional in the context of brain metastasis. Furthermore, the percentages of granulocytes and monocytes correlated with metastatic burden in melanoma brain metastases, suggesting a functional role for recruited myeloid cells in facilitating metastatic growth. Interestingly, analysis of the myeloid cell milieu in breast cancer brain metastasis indicated that similarly to melanoma brain metastasis, there was an elevation in recruited monocytes and granulocytes in brain metastases compared with normal brains. However, the recruitment of monocytes and granulocytes in breast cancer brain metastasis was not significantly LCN2-dependent (FIG. 4B).

Interestingly, analysis of gene expression in recruited granulocytes isolated from melanoma or breast cancer brain metastases revealed an upregulation in their expression of an immunosuppressive gene signature in both melanoma and breast cancer brain metastases in WT mice (FIG. 4C,4D). Strikingly, while in melanoma there was no difference in the expression of this immunosuppressive gene signature in LCN2^{-/-} mice (FIG. 4C), in breast cancer brain metastasis LCN2 was necessary for this functional differentiation of recruited granulocytes (FIG. 4D). Thus, while LCN2 was important for the recruitment of myeloid cells in melanoma brain metastasis, their immunosuppressive phenotype was LCN2-dependent in breast cancer, but not in melanoma brain metastasis. Taken together, these findings show that systemic, as well as local LCN2 in the brain activate pro-inflammatory signaling in astrocytes, resulting in recruitment of immunosuppressive myeloid cells to the brain metastatic microenvironment.

### Example 4

LCN2 Signaling from Bone Marrow-Derived Granulocytes Plays a Key Functional Role in Facilitating Brain Metastasis

The above findings indicated that recruited granulocytes are the main source of LCN2 in brain metastasis. Adoptive bone marrow transplantation (BMT) from WT mice or LCN2^{-/-} mice into lethally irradiated WT mice was performed. Following BMT, mice were injected with melanoma cells and analyzed for brain metastasis, systemic levels of LCN2, and myeloid cell composition in the brain metastatic microenvironment (FIG. 5A). Analysis of LCN2 blood levels one or two weeks following BMT (prior to melanoma cell injection) revealed that basal LCN2 was abolished in the blood of mice transplanted with BM from LCN2^{-/-} mice, confirming that BM-derived cells are the main source of LCN2 (FIG. 5B). Mice were then injected with melanoma cells and followed up for brain metastases. Strikingly, MRI imaging and metastatic burden analysis revealed that mice transplanted with BM from LCN2^{-/-} mice had significantly less brain metastases, confirming that LCN2 from BM-recruited cells is instrumental to support brain metastasis (FIG. 5C-E). Notably, analysis of LCN2 blood levels at end-stage revealed a slight elevation compared with analysis at day 14 (FIG. 5F), consistent with the findings that brain endothelial cells are also a source of LCN2 in brain metastasis. Analysis of myeloid cells in brain metastasis of mice transplanted with WT or LCN2^{-/-} BM indicated enhanced recruitment of monocytes and granulocytes to brain metastasis of mice transplanted with WT BM, which was significantly reduced in mice transplanted with LCN2^{-/-} BM (FIG. 5G). Thus, LCN2 is important for recruitment of granulocytes and monocytes to brain metastases, and recruited LCN2-expressing granulocytes play a central functional role in brain metastatic progression.

### Example 5

LCN2 is a Prognostic Marker in Human Brain Metastasis

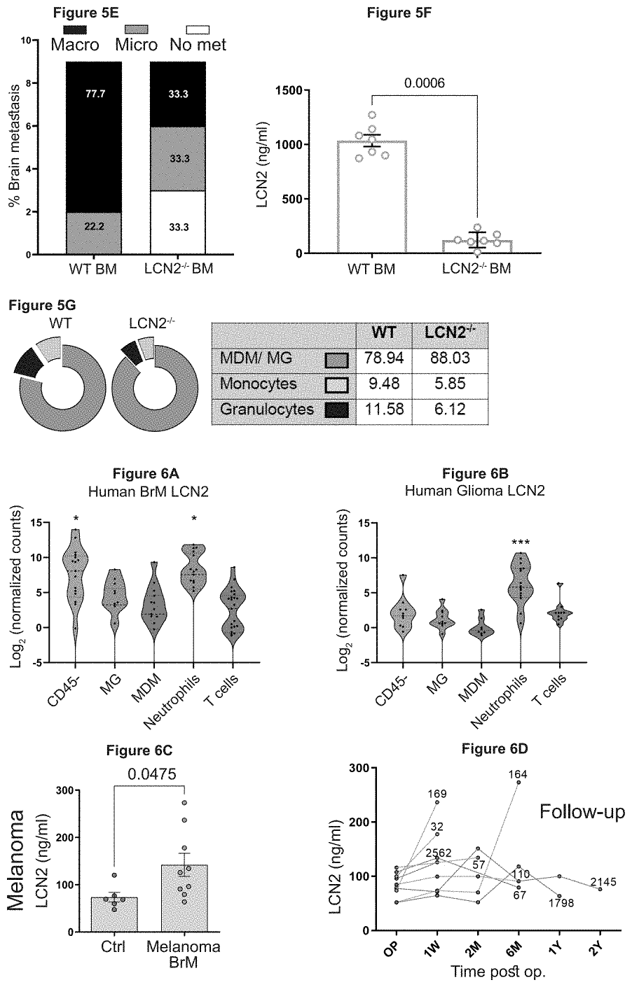

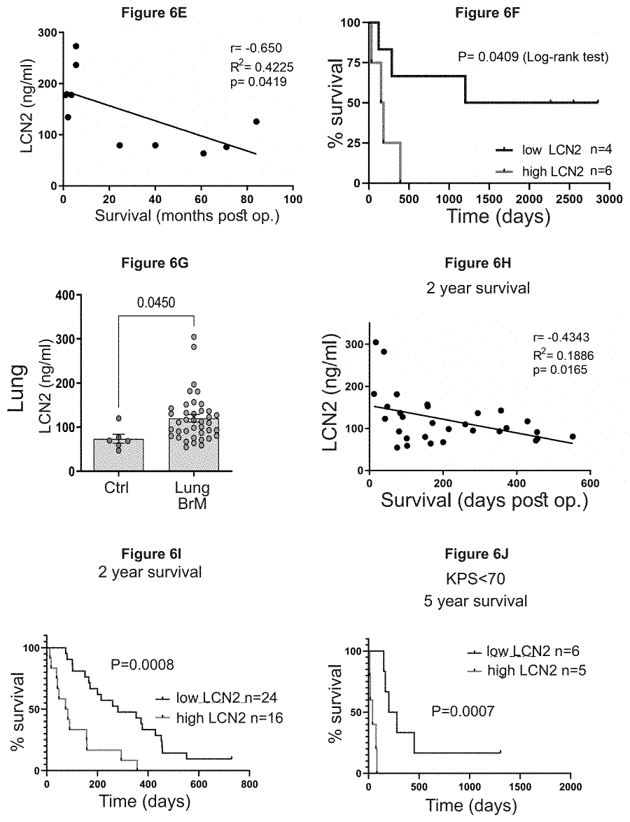

LCN2 signaling was analyzed in human patients with brain metastasis from melanoma, lung and breast cancer, which are amongst the main sources for brain metastasis in patients. Analysis of a dataset of gene expression in human brain metastasis and primary brain tumors confirmed that granulocytes are the main source of LCN2 in the human metastatic microenvironment (FIG. 6A, 6B). Furthermore, in human brain metastasis, CD45⁻ cells were also a significant source of LCN2 in the brain, compatible with its expression in endothelial cells (FIG. 6A).

The systemic levels of LCN2 in patients with brain metastases from melanoma or lung carcinoma was assessed. Analysis of LCN2 in the blood of melanoma patients with brain metastasis confirmed that it was significantly elevated compared with healthy controls (FIG. 6C). Importantly, this cohort of patients included a longitudinal follow-up of blood samples from melanoma patients with brain metastasis. Markedly, temporal analysis of individual samples revealed that a prominent increase in LCN2 blood levels closely preceded patient death (FIG. 6D). The correlation between patient survival and LCN2 levels at their last follow-up was analyzed. It was found that lower levels of LCN2 correlated with longer survival (FIG. 6E, 6F). These results implicate systemic LCN2 as a potential patient follow-up and prognostic marker.

An additional cohort of patients with brain metastases from lung cancer was analyzed to assess whether LCN2 may also function as a prognostic marker in other cancer types. Analysis of blood samples collected from lung cancer patients before surgical removal of their brain metastases confirmed that similarly to what was observed in melanoma metastases, LCN2 levels were higher in the blood of patients compared with healthy controls (FIG. 6G). The correlation of the pre-operative blood levels of LCN2 with patient survival was analyzed. Interestingly, while long-term follow up indicated that the correlation of LCN2 levels with 5-year survival was not significant (not shown), analysis of 2-year survival rates revealed that high levels of LCN2 significantly correlated with worse survival (FIG. 6H, 6I). Importantly, clinical decisions on brain metastasis patient care often have to integrate multiple factors to determine prognosis and treatment paradigms. The clinical disease progress of these patients was investigated by analyzing their Karnofsky Performance Score (KPS). This score is clinically used to quantify the ability of patients to perform in daily life activities and assess their general well-being. The score ranges from 100 (fully active) to 0 (unresponsive). As expected, a lower KPS score correlated with worse survival in the cohort that was analyzed (not shown). However, a subpopulation of patients with low KPS score may still benefit from aggressive treatment rather than palliative care. Thus, stratifying this patient group with additional markers may better define the most beneficial treatment strategy. Notably, stratification of patients with low KPS score (<70) according to their LCN2 levels reveled that high LCN2 was significantly correlated with poor survival (FIG. 6J). Thus, blood levels of LCN2 are linked with disease progression and outcome and provide a novel tool to instruct clinical decision on patient care in fragile patients. Taken together, these findings suggest that systemic levels of LCN2 can be clinically used as a prognostic marker in the management of brain metastatic disease.

### Example 6

Treatment with Simvastatin or Neutralizing Antibody for LCN2 Attenuates Activation of Astrocytes by Melanoma Conditioned Media (CM)

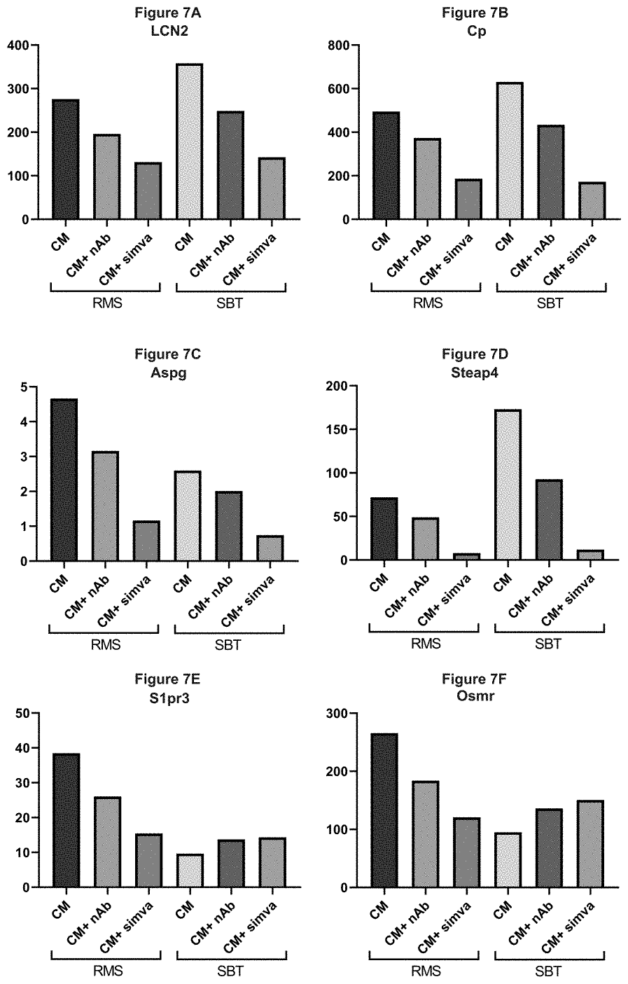

Statins were shown to suppress downstream pathways of LCN2-mediated astrocyte activation including JAK2-STAT3 and Rho-ROCK, and therefore represent a promising therapeutic approach. In order to test in vitro whether LCN2 signaling is responsible for educating astrocytes by melanoma CM, and to assess whether blocking this signaling pathway could attenuate activation of astrocytes, the following experiment was performed: primary astrocytes were cultured with either Ret mCherry sortd (RMS) or spontaneous brain tropic (sBT) CM for 24 h, together with LCN2 neutralizing antibody or with simvastatin. Activation of astrocytes was assessed by analyzing the expression of a pan reactive astrocyte gene signature. Analysis of the results revealed that melanoma CM upregulated the expression of gliosis-related signature genes (FIGS. 7A-7F). Importantly, this upregulation was attenuated by neutralizing LCN2, and by treatment with simvastatin. These results suggest that blocking LCN2 signaling could attenuate activation of astrocytes and may serve as a new therapeutic approach for preventing metastasis.

### Example 7

Pre-Clinical Studies to Determine the Efficacy of Statins Treatment in Preventing/Inhibiting Brain Metastasis:

To assess the therapeutic window of prevention, pre-clinical trials are performed by utilizing the spontaneous metastasis model and intracardiac experimental metastasis models. Mice are treated at distinct time points: In the spontaneous mouse model, immediately after primary tumor resection (prevention trial), and one month following primary tumor removal, when brain micro-metastases are already forming (intervention trial), to test the potential of treatment in inhibiting metastatic progression. In intracardially injected mice, statins are administered starting immediately following tumor cell injection, daily for two weeks, or starting one week after the injection when metastases are already forming. These experiments explore both the preventive capacity of statins by treating mice upon removal of the primary tumor, and their ability to inhibit the growth of already disseminated cells in the already formed pre-metastatic niche.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for treating or preventing brain metastases comprising the step of administering to a patient in need a composition comprising a therapeutically effective amount of LCN2 Inhibitor, an agent that interferes in systemic LCN2 signaling pathways, an agent that reduces LCN2 expression, or any combination thereof.

2. The method of claim 1, wherein the systemic LCN2 signaling pathways instigate neuroinflammation.

3. The method of claim 1, wherein the LCN2 Inhibitor, the agent that interferes in systemic LCN2 signaling pathways, or the agent that reduces LCN2 expression, inhibits astrocytes activation.

4. The method of claim 1, wherein the agent that interferes in systemic LCN2 signaling pathways is an agent that suppresses downstream pathways of LCN2-mediated astrocyte activation.

5. The method of claim 4, wherein the agent that suppresses downstream pathways of LCN2-mediated astrocyte activation suppresses JAK2-STAT3 and/or Rho-ROCK.

6. The method of claim 5, wherein agent that suppresses JAK2-STAT3 and Rho-ROCK is a statin.

7. The method of claim 1, wherein the LCN2 inhibitor, the agent that interferes in systemic LCN2 signaling pathways and/or the agent that reduces LCN2 expression is a neutralizing antibody, a small molecule inhibitor or an antibody to the receptor, an aptamer, a small interfering RNA, a small internally segmented interfering RNA, a short hairpin RNA, a microRNA, and/or antisense oligonucleotide.

8. The method of claim 1, wherein the patient in need is a patient that suffers from melanoma, cutaneous malignant melanoma, melanoma tumorigenesis, breast cancer, lung cancer, melanoma, prostate cancer, colorectal cancer, bladder cancer, bone cancer, blood cancer, thyroid cancer, parathyroid cancer, bone marrow cancer, rectal cancer, throat cancer, laryngeal cancer, esophageal cancer, pancreatic cancer, gastric cancer, tongue cancer, skin cancer, brain tumor, uterine cancer, head or neck cancer, gallbladder cancer, oral cancer, colon cancer, anal cancer, central nervous system tumor, liver cancer, renal cell carcinoma, or colorectal cancer.

9. A method for identifying the suitability of a candidate compound or molecule that inhibits LCN2, interferes in systemic LCN2 signaling pathways, or reduces LCN2 expression, for treating brain metastasis comprising: contacting primary astrocytes with either Ret mCherry sorted (RMS) or spontaneous brain tropic (sBT) conditioned medium with or without the candidate compound or molecule; and comparing activity of astrocytes, wherein if the candidate compound or molecule reduces astrocytes activation in comparison to a control the candidate compound or molecule is suitable for treating the brain metastases.

10. (canceled)

11. A method of determining the survival of a subject afflicted with melanoma-, breast cancer, or lung cancer, comprising the step of comparing LCN2 levels in a blood of the subject with LCN2 levels of a normal healthy subject and/or severe patients, wherein overexpression of the LCN2 beyond the levels of a healthy normal subject determines that the patient has poor survival and overexpression of the LCN2 at the levels of severely ill melanoma, breast cancer or lung cancer patients with low survival determines that the survival of a subject is similar to the survival of severely ill melanoma, breast cancer or lung cancer patients.

12. The method of claim 6, wherein the statin is simvastatin.

13. The method of claim 7, wherein the LCN2 inhibitor is a neutralizing antibody.
